(19) United States
(12) Patent Application Publication
(10) Pub. No.: US 2026/0207101 A1
(43) Pub. Date: Jul. 23, 2026

(54) A DEVICE FOR FLUID TRANSFER THROUGH A PLACED CATHETER

(71) Applicant: B. BRAUN MELSUNGEN AG, Melsungen (DE)

(72) Inventors: Hui Kuun TEOH, Penang (MY); Su Yean ONG, Penang (MY); Jia Ying FOO, Penang (MY)

(73) Assignee: B. BRAUN MELSUNGEN AG, Melsungen (DE)

(21) Appl. No.: 19/141,880

(22) PCT Filed: Dec. 28, 2023
(86) PCT No.: PCT/EP2023/087882

§ 371 Date: Jun. 20, 2025

Related U.S. Application Data
(60) Provisional application No. 63/477,699, filed on Dec. 29, 2022.

(51) Int. Cl. A61B 5/15

(52) U.S. Cl. CPC A61B 5/150488; A61B 5/150496; A61B 5/150633; A61B 5/150992

(57) ABSTRACT

Disclosed is a device for fluid transfer through a placed catheter. The placed catheter has an internal diameter and the device comprises a fine catheter having an external diameter that is smaller than the internal diameter. The device further comprises a distal region adapted to connect to a catheter hub attached to the placed catheter, a proximal region for transferring fluid to or from a fluid collection device, and a contractible envelope extending from the distal region to the proximal region. The fine catheter is being disposed within the envelope and connected to the proximal region so that advancing the proximal region towards the distal region contracts the envelope and advances the fine catheter into the placed catheter.

### TECHNICAL FIELD

The present invention relates, in general terms, to devices and methods for transferring fluid through a placed catheter. The devices may be, although not exclusively, with catheters occluded, for example, by fibrin growth.

### BACKGROUND

It is common in medical treatments, that a patient will require periodical blood tests. This usually involves either repetitive insertion of new needles into the patient, or insertion of a catheter into the vasculature of the patient. In the case of a catheter, the catheter is attached to a catheter hub on the arm or other limb of the patient, and blood is taken by attaching a blood collection container to the catheter hub.

Due to the natural clotting process, fibrin can build up on the tip of the catheter. The fibrin build-up can occlude the catheter, making it more difficult to take blood. This difficulty can result in the use of greater force to draw blood. The greater force can result in haemolysis or kinking of the catheter. Also, where the tip of the catheter is against a vascular wall, the force can cause the catheter tip to suck against the wall.

In addition, greater force applied to a catheter hub increases the risk of dislodgment, leakage, infiltration of contaminants, phlebitis, discomfort to the patient, accidental needle stick injuries and tissue damage.

To extract blood despite the presence of a fibrin deposit, some devices insert a small catheter through the catheter hub and the placed catheter—the catheter already inserted into the vasculature. The small catheter either bypasses the fibrin deposit or punches through it so the tip of the small catheter protrudes from the tip of the placed catheter, into the vasculature and into direct contact with the blood. However, these devices—see, for example, US2017/0216564—are elongated, rigid bodies attached to the catheter hub. While such devices enable controlled insertion of a small catheter through the placed catheter, the forces applied to the elongated, rigid body of such a device is transferred to the catheter hub. This may cause dislodgment, mechanical phlebitis, tissue damage at the puncture site and other problems.

It would be desirable to overcome or ameliorate at least one of the above-described problems, or at least to provide a useful alternative.

### SUMMARY

Disclosed herein is a device for fluid transfer through a placed catheter having an internal diameter, comprising:
a distal region adapted to connect to a catheter hub attached to the placed catheter;
a proximal region for transferring fluid to or from a fluid collection device;
a contractible envelope extending from the distal region to the proximal region; and
a fine catheter having an external diameter that is smaller than the internal diameter, the fine catheter being disposed within the envelope and connected to the proximal region so that advancing the proximal region towards the distal region contracts the envelope and advances the fine catheter into the placed catheter.

As used herein, the term “envelope” refers to a tube, sheath, encasement and any other shroud, cover or similar, that surrounds the portion of the fine catheter that is between the proximal region and distal region and thus prevents access to that portion. The envelope is capable of contracting by collapsing, by telescoping, and/or by crumbling into folds.

The phrase “a placed catheter having an internal diameter” refers to the internal diameter of the placed catheter rather than of the device disclosed herein.

Advancing the proximal region towards the distal region may contract the envelope and advance the fine catheter through the placed catheter so that a distal tip of the fine catheter protrudes from the placed catheter, into the vasculature of a patient. The fine catheter can be fixed to the proximal region so that the fine catheter moves with the proximal region when the proximal region is moved and moves relative to the distal region when the proximal region moves towards the distal region.

The envelope may be hermetically sealed to each of the proximal region and the distal region. Thus, while air or fluid may enter the envelope through other locations—e.g., through an opening in the distal region—air cannot pass through the seal or join between the envelope and proximal region and distal region.

The envelope may comprise a flexible material that contracts by collapsing as the proximal region is advanced towards the distal region.

In some examples, the envelope may comprise a first hollow body and a second hollow body, the second hollow body contracting into the first hollow body as the envelope contracts. The envelope may be extendable to retract the fine catheter from the placed catheter, the second hollow body telescoping from the first hollow body as the envelope extends. The device may also comprise a length index for providing an indication of a length of the fine catheter that has been advanced into the placed catheter, the length index being provided on one or both of the first hollow body and the second hollow body. The length index may alternatively be located elsewhere on the device—e.g., on the fine catheter. The length index may comprise a protrusion and a recess provided on the device—e.g., on respective ones of the first hollow body and second hollow body, the recess engaging the protrusion to provide haptic feedback of the length of the fine catheter that has been advanced into the placed catheter. In this sense, ‘feedback’ is feedback to a user of the device such as a clinician or nurse. In some examples, the envelope may incorporate more than two hollow bodies that telescope.

The distal tip may extend through an aperture in the distal region during contraction of the envelope, and the envelope is extendable so that the distal tip moves proximally of the distal region and out of the aperture.

The device for the transfer of fluid may further comprise a length index for providing to a user an indication of a length of the fine catheter that has been advanced into the placed catheter. The length index may comprise an enlarged or engorged diameter of the fine catheter that abuts an abutment, such as a surface or a shoulder, such that the proximal region cannot be advanced further towards the distal region—e.g., the fine catheter may advance through an aperture in the distal region that is sized to permit passage of the normal diameter of the fine catheter, but is too small to admit passage of the enlarged or engorged diameter portion. The length index may also, or instead, comprises at least two length indices, each indicating a different length of the fine catheter that has been advanced into the placed catheter. If it includes an abutment, the abutment can define a position of full insertion of the fine catheter into or through the placed catheter.

The device may further comprise an extension guide for indicating when the second hollow body is fully extended from the first hollow body. The extension guide may comprise a protrusion and a recess provided on respective ones of the first hollow body and second hollow body, the recess engaging the protrusion to provide haptic feedback that the fine catheter has been fully retracted from the placed catheter. The extension guide may comprise a protrusion and a recess provided on a first body, the first body being one of the first hollow body and second hollow body, and a protrusion provided on a second body, the second body being another of the first hollow body and second hollow body. In use, the protrusion of the first hollow body and second hollow body may move away from each other as the envelope is contracted, and approach each other as the envelope is extended. The protrusion of the second hollow body may abut the protrusion of the first hollow body during extension of the envelope and move past the protrusion of the first body into the recess to fix the first hollow body and second hollow body in a fully extended condition.

The distal region may comprise at least one of:
a male luer connector for connecting to a female luer of the catheter hub;
a female luer connector for connecting to a male luer of the catheter hub; and
a spin-lock fitting for threaded engagement with the catheter hub.

The proximal region may comprise a needle tip for connecting to a fluid collection container—e.g., a vacutainer—and/or at least one of:
a male luer connector for connecting to a female luer of the fluid collection container;
a female luer connector for connecting to a male luer of the fluid collection container; And
a holder for receiving the vacutainer.

The proximal region may comprise a needle with a needle tip and an envelope covering the needle tip, the needle tip puncturing the envelope during connection of a fluid collection container. The needle tip can also puncture through a rubber septum located on a vacutainer.

Disclosed herein is also a device for fluid transfer through a placed catheter having an internal diameter, the device comprising:
a distal region adapted to connect to a catheter hub attached to the placed catheter;
a proximal region for transferring fluid to or from a fluid collection device;
a fine catheter having an external diameter that is smaller than the internal diameter, the fine catheter connected to the proximal region so that advancing the proximal region towards the distal region advances so that a distal tip of the fine catheter protrudes from the placed catheter;
   wherein the device is configured to trap the distal tip within the device after the distal tip moves proximally of the distal region.

Advantageously, by making the envelope contractible, such as by collapsing envelope or telescoping of one part of the envelope into another, the length and the rigidity of the device is reduced when compared with some prior art devices. This reduces the likelihood of mechanical phlebitis and enhances the flexibility of handling during the procedure.

Advantageously, by enabling the tip of the fine catheter to be retracted to a position proximal of the distal region, the tip can become trapped within the envelope and thereby prevent inadvertent reuse of the device.

### BRIEF DESCRIPTION OF THE DRAWINGS

Embodiments of the present invention will now be described, by way of non-limiting example, with reference to the drawings in which:

FIG. 1 is a side view of a device according to an embodiment of the invention;

FIG. 2 is a cross-section view of the device of FIG. 1;

FIG. 3 is a cross-section view of the device of FIG. 1 in a contracted condition, attached to a catheter hub;

FIG. 4 illustrates components for forming a device according to an embodiment of the invention;

FIGS. 5 and 6 illustrate the components of FIG. 4, in the context of assembly thereof into a device according to the present teachings;

FIG. 7, comprising FIGS. 7A to 7D, shows progressive steps of use of the device of FIG. 1;

FIGS. 8A, 8B and 9 are partial views of alternative embodiments of a device in accordance with present teachings;

FIG. 10 is a side perspective view of an alternative embodiment of a device according to the invention;

FIG. 11 is a cross-section view of the device of FIG. 10;

FIG. 12, comprising FIGS. 12A to 12E, shows progressive steps of use of the device of FIG. 11;

FIG. 13 is a cross-section view of an alternative embodiment of a device in accordance with present teachings; and

FIGS. 14 and 15 are cross-section views of alternative embodiments of a device in accordance with present teachings.

### DETAILED DESCRIPTION

The detailed description set forth below in connection with the appended drawings is intended as a description of the presently preferred embodiments of devices and methods for transferring fluid through a placed catheter provided in accordance with aspects of the present invention and is not intended to represent the only forms in which the present devices, systems, and methods may be constructed or utilized. The description sets forth the features and the steps for constructing and using the embodiments of the present devices, systems, and methods in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features.

Descriptions of technical features or aspects of an exemplary configuration of the disclosure should typically be considered as available and applicable to other similar features or aspects in another exemplary configuration of the disclosure. Accordingly, technical features described herein according to one exemplary configuration of the disclosure may be applicable to other exemplary configurations of the disclosure, and thus duplicative descriptions may be omitted herein.

Devices disclosed herein seek to introduce a fine (i.e. small) catheter into the lumen of a placed catheter, so that the fine catheter can be pushed through or bypass an occlusion of the placed catheter. Moreover, embodiments seek to do this in various ways that reduce the likelihood of mechanical phlebitis and other conditions developing. Fine or small is understood to be relative to the placed catheter and is considered fine or small if the device can pass through the lumen of the placed catheter.

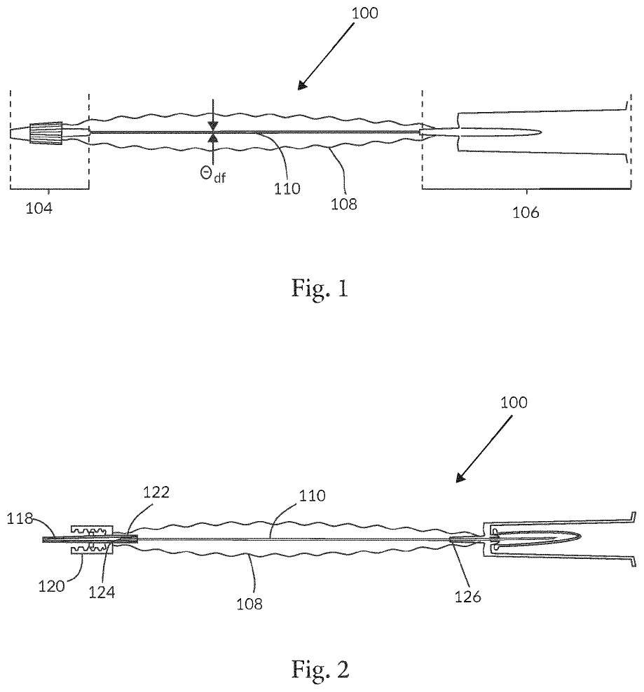

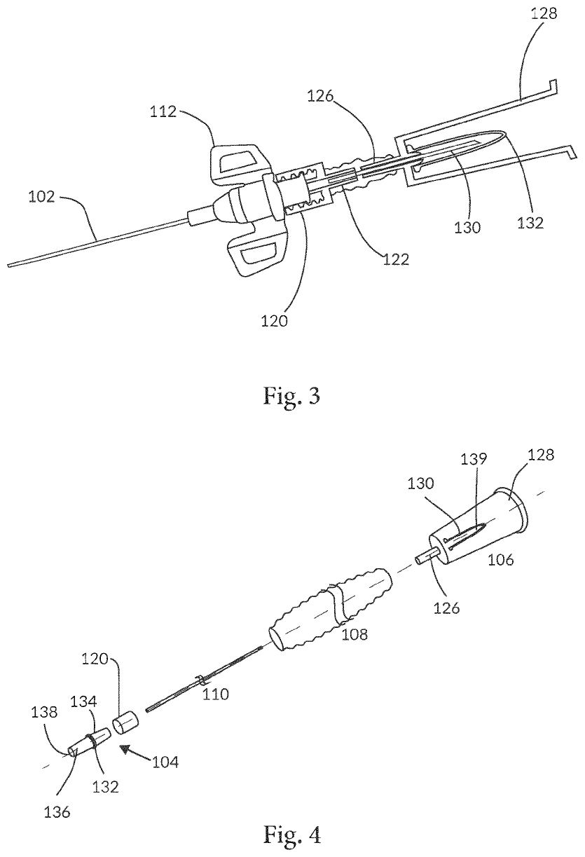

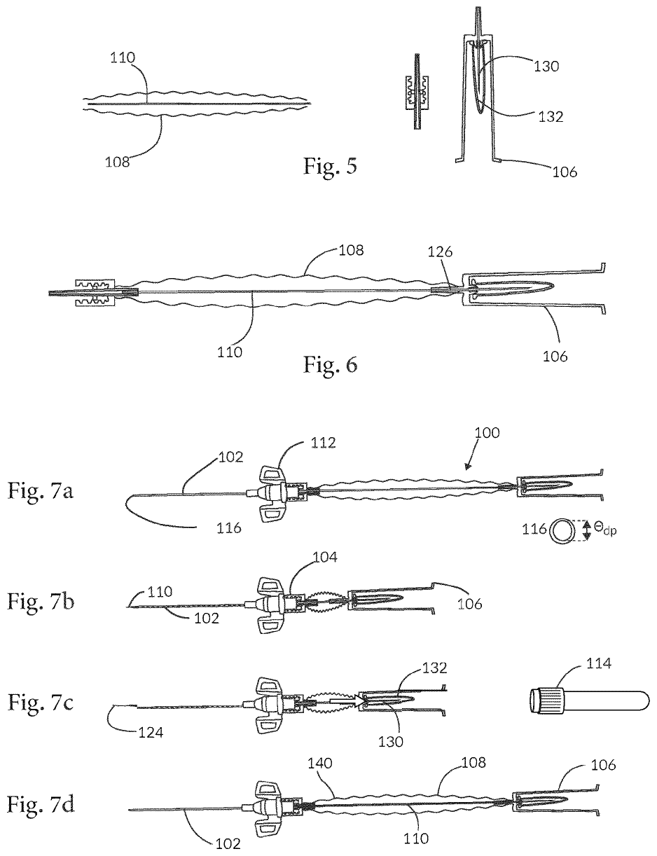

FIG. 1 shows a device 100 for fluid transfer through a placed catheter 102 (see, e.g., FIG. 3). The placed catheter 102 has an internal diameter that may become occluded, for example, by fibrin growth.

The device 100 for transferring fluid through a placed catheter in accordance with aspects of the invention broadly comprises:
a distal region 104;
a proximal region 106;
a contractible envelope 108; and
a fine catheter 110, which has a body with a length and a lumen.

The distal region 104 is adapted to connect to a catheter hub 112 (see, e.g., FIG. 3) that is attached to the placed catheter 102. The proximal region 106 is used to transfer fluid—e.g., blood—to or from a fluid collection device—see, e.g., vacutainer 114 FIG. 7C. In general use, when the fine catheter 110 has been advanced through the placed catheter 102 and into the vasculature of a patient, the fluid, i.e., blood, will travel up the fine catheter 110 within the placed catheter 102, through the distal region 104, into the proximal region 106 and into a blood collection container such as a vacutainer. In other embodiments, the proximal region 106 may comprise the vacutainer or another fluid collection device.

In an example and as further discussed below, the distal region 104 comprises a male connector for coupling to the catheter hub of the placed catheter and the proximal region comprises a holder having a needle inside a cavity for puncturing a septum of a vacutainer received therein. The fine catheter 110 is in fluid communication with the needle in the holder and the fine catheter is slidable relative to a lumen of the male connector at the distal rection 104 so that the fine catheter can be advanced into the lumen of the placed catheter.

In other embodiments, the device 100 is used for infusion or delivery of fluid to the patient. The fluid thus travels in the reverse direction to that described above, from a container holding fluid that is either part of, or connected to, the proximal region 106 of the device 100 and into the fine catheter for delivery to the patient.

The contractible envelope 108 extends from the distal region 104 to the proximal region 106. The contractible envelope 108—i.e., an envelope capable of contraction to a shorter length such as by telescopically shortening of collapsing—reduces the force conveyed from the proximal region 106 to the distal region 104, as the proximal region 106 is advanced by a user towards the distal region 104. The envelope 108 may be any appropriate material and, in the present embodiment, is a transparent, compressible, thin plastic sleeve. The envelope has a structure with a length and two ends. One of the two ends can connect to the holder at the proximal region 106 and the other to the male connector at the distal region 104.

The fine catheter 110 has an external diameter Θ_{df} that is smaller than the internal diameter Θ_{dp} of the placed catheter 102. The internal diameter Θ_{dp} is shown in the end view of the tip 116 of the placed catheter 102 shown in FIG. 7A. The fine catheter 110 is disposed within the envelope 108 and connected to the proximal region 106. Advancing the proximal region 106 towards the distal region 104 of the device contracts the envelope 108, since the distal end of the envelope is fixed at the connector of the distal region and advances the fine catheter 110 into the placed catheter 102. While the placed catheter has a length with a lumen, in general, occlusions typically develop at the distal tip 116 of the placed catheter 102. Therefore, in the present context advancing the fine catheter 110 through the placed catheter 102 will usually mean that a distal tip 124 (FIGS. 2 and 7C) of the fine catheter 110 protrudes from the placed catheter 102—i.e., distally of the distal tip 116 of the placed catheter- into the vasculature of a patient.

The fine catheter 110 may be formed from any suitable material, such as a fluorocarbon - due to it being heavy and less slack during use, having low memory and more of an elastic feel when compared to a monofilament—or Nanofil.

FIG. 2 shows the device 100 in cross-section. The distal region 104 includes a male luer fitting 118 adapted to connect to a female luer fitting of the catheter hub 112. In an example, a threaded collar 120 is provided for threaded engagement with the catheter hub. In some examples, the threaded collar is a spin-lock 120 provided to lock or fix the device 100 onto the catheter hub 112. The male luer fitting 118 may also lock into the spin-lock 120 as shown, or may be otherwise formed with or attached to the spin-lock 120.

It will be appreciated that the distal region 104 can be configured for attachment to any desired catheter hub and is not limited by the embodiment shown. For example, the distal region 104 may comprise at least one of, a male luer connector 122 as shown in FIG. 3, for connecting to a female luer of the catheter hub 112, a female luer connector for connecting to a male luer of the catheter hub, and a spin-lock fitting 120 as shown in FIG. 3, for attaching to a corresponding spin-lock fitting of the catheter hub 112 in a known manner. However, the distal region is not limited to luer-type fittings or locks and can be adapted to connect to any non-Luer type fitting.

Similarly, the proximal region 106 may comprise at least one of a female luer connector 128 for connecting to a male luer connector (not shown) of a fluid collection container, a female luer connector for connecting to a male luer of a fluid collection container, or any other suitable connection, or a holder for receiving a vacutainer.

When the proximal region 106 is a holder for receiving a vacutainer, the holder can include a needle 130 having a sharp tip for puncturing the septum of the vacutainer and an envelope or rubber sleeve 139 for covering the needle 130. The tip of the needle 130 is configured to puncture the envelope 132 during connection of a fluid collection container (not shown) to then puncture the septum of the fluid collection chamber. In particular, the fluid collection chamber—e.g., a vacutainer or vacuum bottle with rubber septum through which the needle 130 punctures—may be pressed against the sleeve or envelop 132 and advanced towards the needle 130. This causes the needle 130 to pierce through the envelope 132, which otherwise ensures sterility of the needle and needle tip and avoids blood exposure risk, and pierce through the rubber septum of the container. As the vacutainer has a predetermined vacuum or “draw”, blood will freely flow into the fluid collection chamber. In some embodiments, the envelope 132 may be omitted. Other components may also be omitted in some embodiments, such as the connector or holder 128.

At the proximal side or end of the distal region 104, a male luer fitting 122 (FIG. 2) is provided. The envelope 108 is attached—e.g., by adhesion, ultrasonic welding or any other suitable process—to the distal region 104. As shown, the distal end of the envelope is attached to the male luer fitting 122 of the distal region 104. To reduce the likelihood of contaminants being introduced by the fine catheter 110 into the lumen of the placed catheter 102, the seal at the connection between the envelope 108 and the distal region 104, such as with the male luer fitting 122, may be a hermetic seal.

The envelope 108 can attach to the proximal region 106 using similar methods as the attachment with the distal region. In an example, the envelope 108 is attached to a male luer fitting 126 of the proximal region 106, such as a using adhesive, ultrasonic welding, or any suitable means. The attachment may also similarly form a hermetic seal between the envelope 108 and the proximal region 106.

For illustration purposes, FIG. 4 shows components of the device 100 in an exploded view. The distal region 104 includes a fitting 133 having a luer cone 134 and a luer connector 136. The envelope 108 has a first end and a second end with the first configured for connecting to the luer one 134 of the distal connector. The proximal region 106 comprises a male luer cone 126 to which the second end of the envelope 108 connects, and the guide tube or fine catheter 110 is in general in alignment with an aperture 138 through the distal region 104, when the device 100 is assembled. To assemble, the guide tube or fine catheter 110 is first inserted into the envelope 108 as shown in FIG. 5. The envelope is preferably made of a medical grade plastic material. The connector at the distal end, which is double-ended, is attached to a threaded collar 120, such as a spin lock collar. The needle 130 and the envelope 132 are typically pre-assembled with the holder 128 in the proximal region 106, unless the proximal region is a different connector, such as a female luer or a needleless connector. The guide tube or fine catheter 110 is then attached, such as by glueing, to the male luer connector 126 at the proximal end. Thus, the fine catheter 110 is fixed in relation to the proximal region 106 and moves as a unit with the proximal region 106 and both move relative to the fitting 133 at the distal region. The envelope 108 is then attached to the proximal region 106, particularly the male luer connector 126. The guide tube or fine catheter 110 is then inserted into aperture 138 of the fitting 133 and the envelope 108 is then attached, such as by adhesion or ultrasonic welding, to the luer cone 134. The device 100 is thus assembled.

FIGS. 7A to 7D show the device 100 in use with a placed catheter 102 that is connected to a catheter hub 112. When blood collection is needed, or simply to avoid having concern about whether or not there is an occlusion, the device 100 is connected to the catheter hub 112 that is attached to a placed catheter 102, as shown in FIG. 7A. The proximal region 106 of the device 100 is then advanced towards the distal region 104 slide the fine catheter relative to the distal region to then extend the fine catheter 110 out the fitting at the distal region 104 and into the placed catheter 102, shown in FIG. 7B. A fluid collection container 114, such as a vacutainer, is then attached to the holder at the proximal region 106 so that the needle 130 punctures the envelope 132 and then the rubber septum on the fluid collection container to then enter the container 114 to allow fluid communication between the proximal region 106 and the container 114, and also between the container 114 and the vasculature of the patient, as shown in FIG. 7C. After a fluid sample, such as blood, is collected in the container 114, or the contents of the container 114 has been infused to the patient, the proximal region 106 is retracted. This involves moving the proximal region in the proximal direction away from the distal region 104 to extend the envelope 108 from the compressed or contracted configuration and to retract the fine catheter 110 from the placed catheter 102, as shown in FIG. 7D.

With reference again to FIG. 3, the device 100 has a collapsed or contracted condition as shown, in which the proximal region 106 has been advanced towards the distal region 104. In this condition, the fine catheter 110 is fully extended into the placed catheter 102 or through the placed catheter 102 such that the tip 124 of the fine catheter 110 extends distally of the distal opening of the placed catheter, as shown in FIG. 7C. Because the envelope 108 can collapse or buckle in folds upon itself, as shown in FIGS. 1 to 3, the envelope is understood as being formed from a flexible material. The flexibility enables to envelope 108, or the material thereof, to contract by collapsing as the proximal region 106 is advanced towards the distal region 104. This collapsed ensure little, if any, force is transferred from the proximal region 106 to the distal region 104 during advancement of the fine catheter 110 into the placed catheter 102, as compared to the envelope being made of a hard material that does not collapse or is difficult to collapse, as an example.

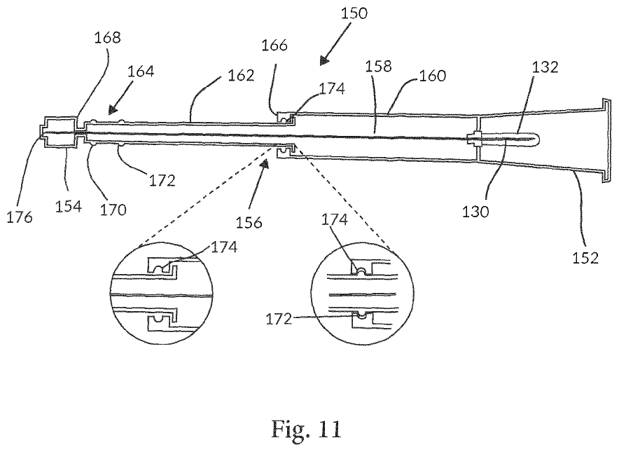

In other embodiments, there may be multiple collapsed or contracted conditions, such as shown in the embodiment in FIG. 11 where indicia demarcate different lengths of extension of the fine catheter 110 into the placed catheter 102 and thus different contracted conditions of the envelope. Aspects of the embodiment of FIG. 11 are further discussed below.

While, after assembly of the device 100 as shown in FIGS. 7A to 7C, the distal tip 124 of the fine catheter 110 extends through the aperture 138 (FIG. 4) in the distal region 104, on extension of the envelope 108 the distal tip 124 moves proximally of the distal region 104. This causes the distal tip 124 to come proximally out of the aperture 138 and fall free within the envelope 108. The tip 124 is thereby trapped within the envelope 108. This configuration prevents inadvertent reuse of the device 100. Other arrangements may also be used, such as a small metal biasing arm 140 (FIG. 7D) (shown in broken lines as it does not form part of the present embodiment but is instead an alternative embodiment) that displaces the fine catheter 110 laterally of the lengthwise axis of the device 100 after the distal tip 124 of the fine catheter moves proximally of the distal region 104, to prevent the fine catheter 110 from re-aligning and entering aperture 138. The small metal biasing arm 140 may apply insufficient force to interfere with the device 100 moving to the contracted condition, but may apply sufficient force to prevent re-entry of the fine catheter 110 into the aperture 138 after it moves proximally thereof.

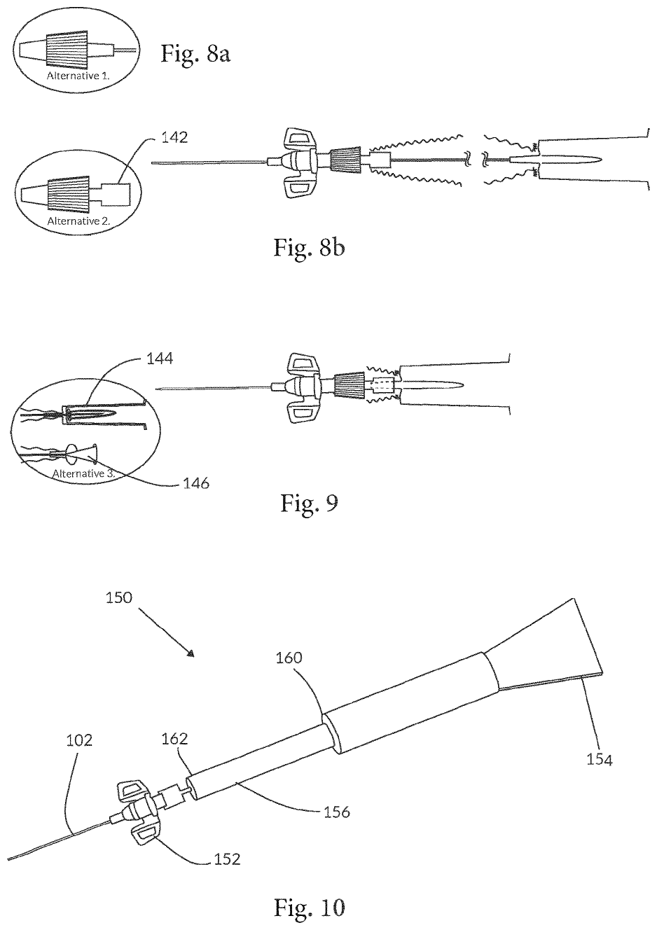

FIG. 8A shows the distal region 104 of the present embodiment, in close-up view. As previously discussed, the distal region 104 of the device 100 comprises a fitting for engaging the catheter hub of the placed catheter and for securement of attachment of the first end of the envelope 108. FIG. 8B shows an alternative embodiment of the distal region 104 employing a female luer connector 142 to which the envelope 108 attaches. These figures illustrate that a variety of different arrangements may be used for the distal region 104 and that the distal region can embody a range of structures for attaching to the catheter hub and the envelope and for allowing the fine catheter to pass therethrough and that these options are all intended to fall within the scope of the present disclosure.

FIG. 9 shows alternative shapes for the proximal region 106 of the device 100. The holder 144 is configured to be used with a fluid collection assembly, such as a vacutainer, and the female connector 146 can be a female Luer connector. Other connector arrangements are contemplated and may be used as desired and fall within the scope of the present invention, such as a needleless connector with a compressible piston located inside for controlling fluid through the body of the needleless connector.

FIG. 10 shows an alternative embodiment of a device 150 for fluid transfer that operates in a substantially similar way to the device 100 discussed elsewhere herein. The device 150 of the present alternative embodiment can be similarly used for fluid transfer through a placed catheter 102. The device 150 broadly comprises:
a distal region 152;
a proximal region 154;
a contractible envelope 156; and
a fine catheter 158 (FIG. 11), which has a body with a length and a lumen.

The envelope 156 may be opaque, as shown, translucent, or transparent. The envelope 156, when compared with the envelope 108 of FIG. 2, is the main difference between the two devices 100, 150. With reference to FIG. 11, the envelope 156 of device 150 comprises a first hollow body 160 and a second hollow body 162. These two bodies 160, 162 can be circular in cross-section and tubular, though any appropriate cross-section may be used as needed. Rather having a single body that collapses like the envelope 108 of the device 100 of FIG. 2 on advancement of the proximal region 106 towards the distal region 104, the second hollow body 162 of the present embodiment contracts into the first hollow body 160 as the envelope 156 contracts in a telescoping manner. In other examples, the second hollow body 162 can go over the first hollow body 160, or the first hollow body can move inside the second hollow body, when the proximal region is advanced towards the distal region.

The telescoping envelope 156 can be extendable from the contracted position to an extended position to retract the fine catheter 158 from the placed catheter 102. In so doing, the second hollow body 162 moves away from the first hollow body 160 as the envelope 156 extends.

In some embodiments, there may be more than two hollow bodies forming the envelope 158, such as three or more hollow bodies forming the contractable envelope. The hollow bodies may be concentrically disposed or arranged in any desired arrangement that facilitates the ease of use and reduces forces application to the catheter hub by the device during use.

The envelope 156 may be integrally formed with one or both of the proximal region 104 and distal region 102, such as by injection moulding. Alternatively, the envelope 156 may be formed separately and attached to the proximal region and the distal region, in similar manners as described above. Many features of device 150 will be understood from the description of device 100 and will not be repeated here.

The device 150 can include a length index 164. The length index 164 is configured to provide to a user an indication of a length of the fine catheter 158 that has been advanced into the placed catheter 102. The length index of FIG. 11 is shown provided on both the first hollow body 160 and second hollow body 162, although only one or the other may be practiced. In the embodiment shown, the device index can be haptic feedback or a ‘click’.

The length index 164 can comprise one or more protrusions, presently two protrusions 170, 172, arranged serially along the length of the second hollow body 162 and a recess 174 provided on the first hollow body 160. As the first and second hollow bodies contract or inwardly telescope upon advancement of the proximal region during use, protrusion serially engages the recess to indicate the length of advancement of the fine catheter that has advanced by the movement. The protrusions may form circumferential ribs or be small localised projections radially outwardly from the body on which they are formed. Similarly, the recess 174 may be circumferential or an annular groove, thereby removing the need to orient the first body 160 relative to the second body 162 during contraction of the envelope 158 to ensure the recess and the protrusion come into engagement.

The length index 164 may similarly comprise more than the two length index components, such as more than two serially placed protrusions 170, 172 along the length of the exterior of the second hollow body, each of which indicates a different length of the fine catheter 158 that has been advanced into the placed catheter 102. If the recess 174 is circumferential or an annular recess, then the multiple protrusions can be located serially along the length of the second hollow body but can be radially located relative to one another and still engage the annular recess during advancement of the proximal region.

The length index may instead simply be an abutment, such as the distal end 166 of the first hollow body 160 coming into abutment with a wall or shoulder portion 168 of the distal region 104 and physically preventing further advancement of the proximal region 154 towards the distal region 152. Thus, the length index in such a case would involve the first hollow body 160 and the distal region 104. In the device 100 of FIGS. 1 and 2, a length index can be incorporated. For example, the length index can involve one or more recesses or protrusions on the distal region 104 that are engaged by a corresponding one or more protrusions or recesses on the proximal region 106, on the envelope 108, or on the exterior of the fine catheter 110.

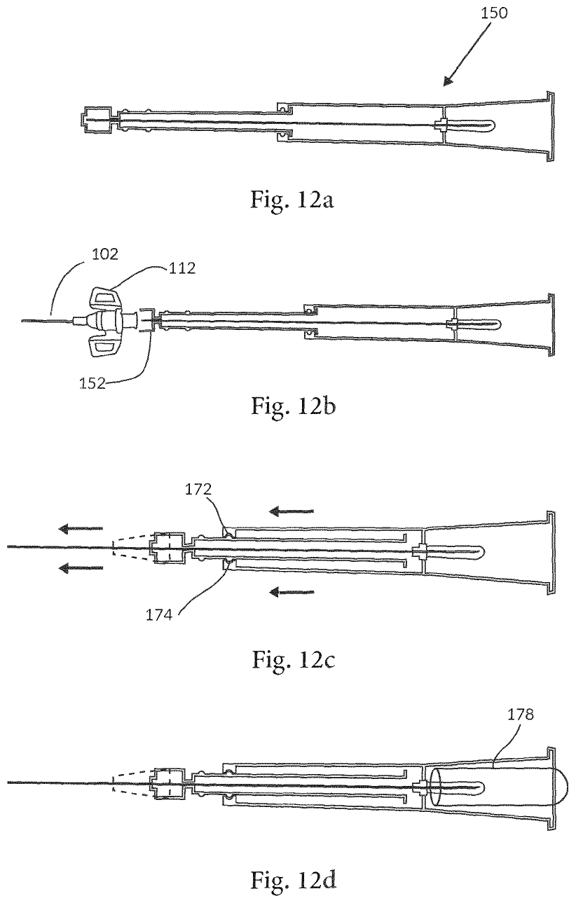

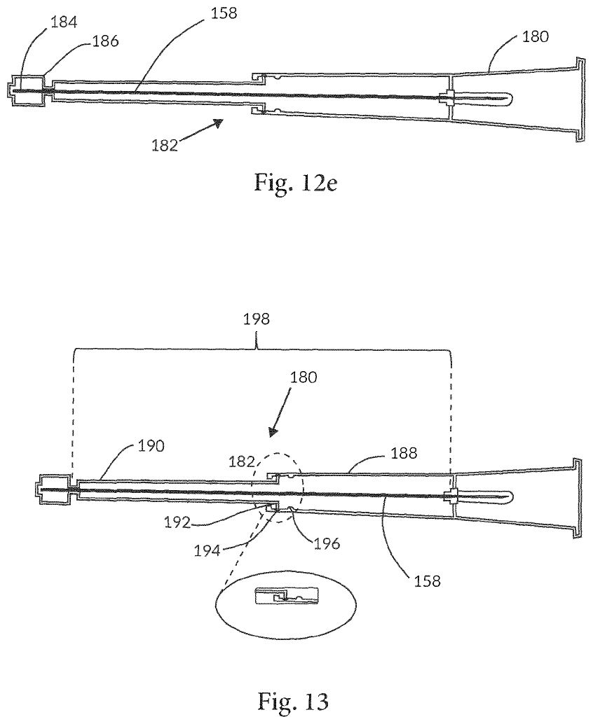

An exemplary use case of the device 150 of FIG. 11 is illustrated in FIGS. 12A to 12E. Firstly, the device 150 is removed from its sterile packaging (FIG. 12A). The device can have a length that is around 200 mm long at this stage, although other lengths are contemplated for different applications. The tip of the fine catheter 158 is located in the lumen of the fitting at the distal region at this stage. The distal region 152 is then connected to the catheter hub 112 that is attached to a placed catheter 102 (FIG. 12B). The proximal region 154 is then advanced towards the distal region 152 to telescopically contract the second hollow body 162 into the first hollow body 160, as shown in FIG. 12C. Thus, the protrusion 172 located on the second hollow body enters recess 174 of the first hollow body (FIG. 11). This provides haptic feedback of the length of the fine catheter 158 that has been advanced into the placed catheter 102.

If the proximal region 154 were advanced further, the recess 174 would move past the second protrusion 172 and over or into engagement with the first protrusion 170. This further advancement provides feedback of further advancement of the fine catheter 158 into the placed catheter by a known distance measured between the first protrusion 170 and the second protrusion 172. A user can interpret the length index in the following examples. For a shorter placed catheter, in the order of about 23 mm, the second protrusion 172 engaging the recess 174 may be suitable to ensure that the distal tip 176 of the fine catheter 158 protrudes past the distal tip of the placed catheter 102 and no further advancement of the proximal region is necessary. For a longer placed catheter, in the order of about 32 mm, the first protrusion 170 engaging the recess 174 may be suitable to indicate that the distal tip of the fine catheter protrudes past the distal tip of the placed catheter.

When in the configuration to suit the approximately 25 mm placed catheter, the device for transferring fluid 150 may be around 142 mm long. Once the proximal region is advanced towards the distal region, a container 178 can attach to the proximal region 154 (FIG. 12D) or the device 150 may comprise a built-in vacutainer or other container. Once the container 178 is filled, or its contents have been infused, the container can be taken away and the envelope 156 returns to its extended configuration. That is, the second body 162 telescopes out from the first body 160. This is shown generally in FIG. 12E, which shows the device 150 with a holder 180 for cooperating with a vacutainer.

The device of FIG. 12E is shown with an extension guide 182. In the configuration of FIG. 12E, the fine catheter 158 is configured to fill the container for use with the device with fluid, such as blood. The tip 184 of the fine catheter 158 may be drawn past the proximal end of the distal region 186 when retracting the proximal region, the same as described above for device 100 to prevent re-use of the device. However, the tip 184 of the fine catheter 158 may instead remain inside the distal region 186 of device by incorporating a telescoping locking feature, as an example.

To prevent reinsertion or reuse of the fine catheter 158 after fluid has infused into the patient or a sample has been taken from the patient, the device 180 of FIG. 13 can incorporate an extension guide 182. In an example, the extension guide 182 can be sized and shaped to indicate when the second hollow body is fully extended from the first hollow body. Further, the extension guide 182 can incorporate structures to prevent inward telescoping movement between the first hollow body 188 and second hollow body 190 after the device 180 has been moved to a fully extended condition as shown, after use.

In the example shown, the extension guide 182 comprises a protrusion 192 and a recess 194 provided on respective ones of the first hollow body 188 and second hollow body 190. The recess 194, which can have an annular groove, engages the protrusion 192 to provide haptic feedback that the fine catheter 158 has been fully retracted from the placed catheter 102.

The present extension guide 182 also comprises a protrusion 196 located on the first hollow body 188. The protrusion 196 on the first hollow body 188 provides an indication, presently haptic, that the device 180 is about to enter a fully extended state since the envelope of the present device incorporates a lock and cannot again inwardly telescope once fully extended and locked. This reduces the likelihood of inadvertent and irreversible extension of the device 180 to a fully extended condition as shown in FIG. 13, in which protrusion 192 is irreversibly trapped in the recess 194 and serves as a telescoping lock. As a result, in use, the protrusion 196 located on the first hollow body 188 and the protrusion 192 located on the second hollow body 190 move away from each other as the envelope 198 is contracted, and approach each other as the envelope 198 is extended in FIG. 13. During extension, the protrusion 192 of the second hollow body 190 moves past the protrusion 196 of the first hollow body 188 and into the recess 194 to fix the first hollow body 188 and second hollow body 190 in a fully extended condition.

It will be understood that the protrusions and recesses may be provided on the other one of the first hollow body 188 and second hollow body 190 without detracting from the operation of the extension guide. It will further be understood that the engagement of protrusions and recesses, or other mechanism as applied in a particular embodiment, may lock the relative positions of the proximal region and distal region (e.g., by locking portions of the envelope or sheath) thereby preventing reuse of the device.

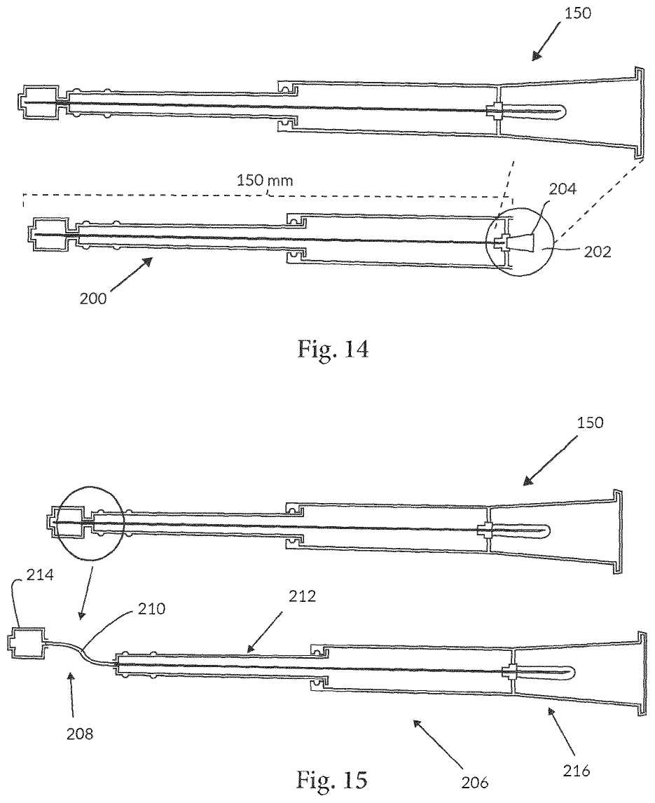

FIG. 14 shows a device 200 for transferring fluid in accordance with further aspects of the invention, shown adjacent the device 150 of FIG. 13 for discussion purposes. In the present embodiment, the device 200 has a proximal region 202 having a small adapter 204 for connection to a blood collection or fluid infusion container. For example, the small adapter 204 can be a female Luer for receiving a male Luer tip of a syringe.

FIG. 15 shows an alternative embodiment device for transferring fluid 206 located adjacent the device 150 of FIG. 13 for discussion purposes. The device 206 has a distal region 208 comprising a flexible tube 210 extending between the envelope 212 and a connector 214. The connector can be a male threaded luer for connecting to the catheter hub of a placed catheter (not shown), which can optionally be a spin lock collar located about a fitting. The flexible tube 210 can reduce the force applied by the proximal region 216 to the catheter hub during contraction of the envelope 212, such as during advancement of the proximal region towards the distal region. The flexible tube 210 also allows the device 206 the flexibility to be used while at a range of angles relative to the catheter hub, without placing unnecessary forces on the catheter hub that might otherwise pull the placed catheter out of the patient.

Embodiments of the device for transferring fluid with a fine catheter described herein can provide one or more of the functions or advantages of reducing the risk of blood exposure, providing direct feedback of extension and contraction during use, obtaining blood samples with minimal haemolysis, allowing good stability throughout the procedure by reducing force applied to the catheter hub of a placed catheter, and preventing unintentional re-use of the device.

Methods of using the device and making the device are within the scope of the invention.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following are numbered example embodiments of the devices, systems, and methods for transferring fluid inside a placed catheter. Examples 1-42 or any other examples disclosed herein may be combined in whole or in part. Elements of the examples disclosed herein are not limiting.

Example 1. A device for fluid transfer through a placed catheter having an internal diameter, comprising: a distal region adapted to connect to a catheter hub attached to the placed catheter; a proximal region for transferring fluid to or from a fluid collection device; a contractible envelope extending from the distal region to the proximal region; and a fine catheter having an external diameter that is smaller than the internal diameter, the fine catheter being disposed within the envelope and connected to the proximal region so that advancing the proximal region towards the distal region contracts the envelope and advances the fine catheter into the placed catheter.

Example 2. The system, device, apparatus, and method for fluid transfer of any of Examples 1-42 or any other embodiment described herein, wherein the envelope has a first length when extended and a second length, which is shorter than the first length, when contracted, which corresponds to when the fine catheter advances through the placed catheter so that a distal tip of the fine catheter protrudes from a distal opening of the placed catheter.

Example 3. The system, device, apparatus, and method for fluid transfer of any of Examples 1-42 or any other embodiment described herein, wherein the envelope is hermetically sealed to the proximal region and to the distal region.

Example 4. The system, device, apparatus, and method for fluid transfer of any of Examples 1-42 or any other embodiment described herein, wherein the envelope comprises a flexible material that contracts by collapsing as the proximal region is advanced towards the distal region.

Example 5. The system, device, apparatus, and method for fluid transfer of any of Examples 1-42 or any other embodiment described herein, wherein the envelope comprises at least a first hollow body and a second hollow body that telescope.

Example 6. The system, device, apparatus, and method for fluid transfer of any of Examples 1-42 or any other embodiment described herein, wherein the second hollow body is configured to contract into the first hollow body when the proximal region advances towards the distal region.

Example 7. The system, device, apparatus, and method for fluid transfer of any of Examples 1-42 or any other embodiment described herein, wherein the envelope is extendable from a contracted state to an extended state to retract the fine catheter from the placed catheter, and wherein the second hollow body telescoping outwardly from the first hollow body as the envelope extends.

Example 8. The system, device, apparatus, and method for fluid transfer of any of Examples 1-42 or any other embodiment described herein, wherein the envelope is extendable to retract the fine catheter from the placed catheter and the device is configured to trap the distal tip within the device after the distal tip moves proximally of the distal region.

Example 9. The system, device, apparatus, and method for fluid transfer of any of Examples 1-42 or any other embodiment described herein, wherein the distal tip extends through an aperture in the distal region during contraction of the envelope, and the envelope is extendable so that the distal tip moves proximally of the distal region and out of the aperture.

Example 10. The system, device, apparatus, and method for fluid transfer of any of Examples 1-42 or any other embodiment described herein, further comprising a length index for providing an indication of a length of the fine catheter that has been advanced into the placed catheter.

Example 11. The system, device, apparatus, and method for fluid transfer of any of Examples 1-42 or any other embodiment described herein, further comprising a length index for providing an indication of a length of the fine catheter that has been advanced into the placed catheter, the length index being provided on one or both of the first hollow body and the second hollow body.

Example 12. The system, device, apparatus, and method for fluid transfer of any of Examples 1-42 or any other embodiment described herein, wherein the length index comprises a protrusion and a recess provided on respective ones of the first hollow body and second hollow body, the recess engaging the protrusion to provide haptic feedback of the length of the fine catheter that has been advanced into the placed catheter.

Example 13. The system, device, apparatus, and method for fluid transfer of any of Examples 1-42 or any other embodiment described herein, wherein the length index comprises at least one of: an abutment such that the proximal region cannot be advanced further towards the distal region; and at least two length indices, each indicating a different length of the fine catheter that has been advanced into the placed catheter.

Example 14. The system, device, apparatus, and method for fluid transfer of any of Examples 1-42 or any other embodiment described herein, further comprising an extension guide for indicating when the second hollow body is fully extended from the first hollow body.

Example 15. The system, device, apparatus, and method for fluid transfer of any of Examples 1-42 or any other embodiment described herein, wherein the extension guide comprises a protrusion and recess provided on respective ones of the first hollow body and second hollow body, the recess engaging the protrusion to provide haptic feedback that the fine catheter has been fully retracted from the placed catheter.

Example 16. The system, device, apparatus, and method for fluid transfer of any of Examples 1-42 or any other embodiment described herein, wherein the extension guide comprises a protrusion and recess provided on a first body, the first body being one of the first hollow body and second hollow body, and a protrusion provided on a second body, the second body being another of the first hollow body and second hollow body.

Example 17. The system, device, apparatus, and method for fluid transfer of any of Examples 1-42 or any other embodiment described herein, wherein, in use, the protrusion of the first hollow body and second hollow body move away from each other as the envelope is contracted, and approach each other as the envelope is extended.

Example 18. The system, device, apparatus, and method for fluid transfer of any of Examples 1-42 or any other embodiment described herein, wherein the protrusion of the second hollow body abuts the protrusion of the first hollow body during extension of the envelope, and moves past the protrusion of the first body into the recess to fix the first hollow body and second hollow body in a fully extended condition.

Example 19. The system, device, apparatus, and method for fluid transfer of any of Examples 1-42 or any other embodiment described herein, wherein the distal region comprises at least one of: a male luer connector for connecting to a female luer of the catheter hub; a female luer connector for connecting to a male luer of the catheter hub; and a spin-lock fitting for threaded engagement with the catheter hub.

Example 20. The system, device, apparatus, and method for fluid transfer of any of Examples 1-42 or any other embodiment described herein, wherein the proximal region comprises a needle tip for connecting to a fluid collection container, and at least one of: a male luer connector for connecting to a female luer of the fluid collection container; a female luer connector for connecting to a male luer of the fluid collection container; and a holder for receiving the fluid collection container.

Example 21. The system, device, apparatus, and method for fluid transfer of any of Examples 1-42 or any other embodiment described herein, wherein the proximal region comprises a needle having a needle tip and an envelope covering the needle tip, the needle tip puncturing the envelope during connection of a fluid collection container.

Example 22. A device for fluid transfer through a placed catheter having an internal diameter, the device comprising: a distal region adapted to connect to a catheter hub attached to the placed catheter; a proximal region for transferring fluid to or from a fluid collection device; a fine catheter having an external diameter that is smaller than the internal diameter, the fine catheter connected to the proximal region so that advancing the proximal region towards the distal region advances the fine catheter so that a distal tip of the fine catheter protrudes out of the placed catheter; wherein the device is configured to trap the distal tip within the device after the distal tip moves proximally of the distal region following use.

Example 23. The system, device, apparatus, and method for fluid transfer of any of Examples 1-42 or any other embodiment described herein, further comprising: a contractible envelope extending from the distal region to the proximal region; the fine catheter being disposed within the envelope and connected to the proximal region so that advancing the proximal region towards the distal region contracts the envelope and advances the fine catheter into the placed catheter.

Example 24. The system, device, apparatus, and method for fluid transfer of any of Examples 1-42 or any other embodiment described herein, The device of claim 23, wherein the envelope is hermetically sealed to the proximal region and to the distal region.

Example 25. The system, device, apparatus, and method for fluid transfer of any of Examples 1-42 or any other embodiment described herein, wherein the envelope comprises a flexible material that contracts by collapsing as the proximal region is advanced towards the distal region.

Example 26. The system, device, apparatus, and method for fluid transfer of any of Examples 1-42 or any other embodiment described herein, wherein the envelope comprises at least a first hollow body and a second hollow body, the second hollow body contracting into the first hollow body as the envelope contracts.

Example 27. The system, device, apparatus, and method for fluid transfer of any of Examples 1-42 or any other embodiment described herein, wherein the envelope is extendable to retract the fine catheter proximally from the placed catheter, and wherein the second hollow body telescope outwardly from the first hollow body as the envelope extends.

Example 28. The system, device, apparatus, and method for fluid transfer of any of Examples 1-42 or any other embodiment described herein, wherein the distal tip extends through an aperture in the distal region during contraction of the envelope, and the envelope is extendable so that the distal tip moves proximally of the distal region and out of the aperture.

Example 29. The system, device, apparatus, and method for fluid transfer of any of Examples 1-42 or any other embodiment described herein, further comprising a length index for providing an indication of a length of the fine catheter that has been advanced into the placed catheter.

Example 30. The system, device, apparatus, and method for fluid transfer of any of Examples 1-42 or any other embodiment described herein, further comprising a length index for providing an indication of a length of the fine catheter that has been advanced into the placed catheter, the length index being provided on one or both of the first hollow body and the second hollow body.

Example 31. The system, device, apparatus, and method for fluid transfer of any of Examples 1-42 or any other embodiment described herein, wherein the length index comprises a protrusion and a recess provided on respective ones of the first hollow body and second hollow body, the recess engaging the protrusion to provide haptic feedback of the length of the fine catheter that has been advanced into the placed catheter.

Example 32. The system, device, apparatus, and method for fluid transfer of any of Examples 1-42 or any other embodiment described herein, wherein the length index comprises at least one of: an abutment such that the proximal region cannot be advanced further towards the distal region; and at least two length indices, each indicating a different length of the fine catheter that has been advanced into the placed catheter.

Example 33. The system, device, apparatus, and method for fluid transfer of any of Examples 1-42 or any other embodiment described herein, further comprising an extension guide for indicating when the second hollow body is fully extended from the first hollow body.

Example 34. The system, device, apparatus, and method for fluid transfer of any of Examples 1-42 or any other embodiment described herein, wherein the extension guide comprises a protrusion and recess provided on respective ones of the first hollow body and second hollow body, the recess engaging the protrusion to provide haptic feedback that the fine catheter has been fully retracted from the placed catheter.

Example 35. The system, device, apparatus, and method for fluid transfer of any of Examples 1-42 or any other embodiment described herein, wherein the extension guide comprises a protrusion and recess provided on a first body, the first body being one of the first hollow body and second hollow body, and a protrusion provided on a second body, the second body being another of the first hollow body and second hollow body.

Example 36. The system, device, apparatus, and method for fluid transfer of any of Examples 1-42 or any other embodiment described herein, wherein, in use, the protrusion of the first hollow body and second hollow body move away from each other as the envelope is contracted, and approach each other as the envelope is extended.

Example 37. The system, device, apparatus, and method for fluid transfer of any of Examples 1-42 or any other embodiment described herein, wherein the protrusion of the second hollow body abuts the protrusion of the first hollow body during extension of the envelope, and moves past the protrusion of the first body into the recess to fix the first hollow body and second hollow body in a fully extended condition.

Example 38. The system, device, apparatus, and method for fluid transfer of any of Examples 1-42 or any other embodiment described herein, wherein the distal region comprises at least one of: a male luer connector for connecting to a female luer of the catheter hub; a female luer connector for connecting to a male luer of the catheter hub; and a spin-lock fitting for threaded engagement with the catheter hub.

Example 39. The system, device, apparatus, and method for fluid transfer of any of Examples 1-42 or any other embodiment described herein, wherein the proximal region comprises a needle having a needle tip for connecting to a fluid collection container, and at least one of: a male luer connector for connecting to a female luer of the fluid collection container; a female luer connector for connecting to a male luer of the fluid collection container; and a holder for receiving the fluid collection container.

Example 40. The system, device, apparatus, and method for fluid transfer of any of Examples 1-42 or any other embodiment described herein, wherein the proximal region comprises a needle having a needle tip and an envelope covering the needle tip, the needle tip puncturing the envelope during connection of a fluid collection container.

Example 41. A method of forming a device for fluid transfer through a placed catheter having an internal diameter, comprising: forming a distal region, the distal region is adapted to connect to a catheter hub attached to the placed catheter; forming a proximal region, the proximal region having a structure for transferring fluid to or from a fluid collection device; locating a contractible envelope between the distal region and the proximal region and securing the envelope to the proximal and distal regions; and connecting a fine catheter to the proximal region, the fine catheter having an external diameter that is smaller than the internal diameter and being disposed within the envelope; and wherein the envelope contracts when the proximal region advances towards the distal region.

Example 42. The system, device, apparatus, and method for fluid transfer of any of Examples 1-42 or any other embodiment described herein, wherein the fine catheter is movable relative to the distal region when the proximal region advances towards the distal region.

The reference in this specification to any prior publication (or information derived from it), or to any matter which is known, is not, and should not be taken as an acknowledgment or admission or any form of suggestion that such prior publication (or information derived from it) or known matter forms part of the common general knowledge in the field of endeavor to which this specification relates.

1. A device for fluid transfer through a placed catheter having an internal diameter, comprising:
a distal region adapted to connect to a catheter hub attached to the placed catheter;
a proximal region for transferring fluid to or from a fluid collection device;
a contractible envelope extending from the distal region to the proximal region; and
a fine catheter having an external diameter that is smaller than the internal diameter, the fine catheter being disposed within the envelope and connected to the proximal region so that advancing the proximal region towards the distal region contracts the envelope and advances the fine catheter into the placed catheter.

2. The device of claim 1, wherein the envelope has a first length when extended and a second length, which is shorter than the first length, when contracted, which corresponds to when the fine catheter advances through the placed catheter so that a distal tip of the fine catheter protrudes from a distal opening of the placed catheter.

3. The device of claim 1, wherein the envelope is hermetically sealed to the proximal region and to the distal region.

4. The device of claim 1, wherein the envelope comprises a flexible material that contracts by collapsing as the proximal region is advanced towards the distal region.

5. The device of claim 1, wherein the envelope comprises at least a first hollow body and a second hollow body that telescope.

6. The device of claim 2, wherein the envelope is extendable to retract the fine catheter from the placed catheter and the device is configured to trap the distal tip within the device after the distal tip moves proximally of the distal region.

7. The device of claim 1, further comprising a length index for providing an indication of a length of the fine catheter that has been advanced into the placed catheter.

8. The device of claim 5, further comprising a length index for providing an indication of a length of the fine catheter that has been advanced into the placed catheter, the length index being provided on one or both of the first hollow body and the second hollow body.

9. The device of claim 8, wherein the length index comprises a protrusion and a recess provided on respective ones of the first hollow body and second hollow body, the recess engaging the protrusion to provide haptic feedback of the length of the fine catheter that has been advanced into the placed catheter.

10. The device of claim 5, further comprising an extension guide for indicating when the second hollow body is fully extended from the first hollow body.

11. The device of claim 10, wherein the extension guide comprises a protrusion and recess provided on respective ones of the first hollow body and second hollow body, the recess engaging the protrusion to provide haptic feedback that the fine catheter has been fully retracted from the placed catheter.

12. The device of claim 1, wherein the distal region comprises at least one of:
a male luer connector for connecting to a female luer of the catheter hub;
a female luer connector for connecting to a male luer of the catheter hub; and
a spin-lock fitting for threaded engagement with the catheter hub.

13. The device of claim 1, wherein the proximal region comprises a needle tip for connecting to a fluid collection container, and at least one of:
a male luer connector for connecting to a female luer of the fluid collection container;
a female luer connector for connecting to a male luer of the fluid collection container; and
a holder for receiving the fluid collection container.

14. A method of forming a device for fluid transfer through a placed catheter having an internal diameter, comprising:
forming a distal region, the distal region is adapted to connect to a catheter hub attached to the placed catheter;
forming a proximal region, the proximal region having a structure for transferring fluid to or from a fluid collection device;
locating a contractible envelope between the distal region and the proximal region and securing the envelope to the proximal and distal regions; and
connecting a fine catheter to the proximal region, the fine catheter having an external diameter that is smaller than the internal diameter and being disposed within the envelope; and
wherein the envelope contracts when the proximal region advances towards the distal region.

15. The method of claim 14, wherein the fine catheter is movable relative to the distal region when the proximal region advances towards the distal region.
